(19) United States
(12) Patent Application Publication
(10) Pub. No.: US 2023/0057358 A1
(43) Pub. Date: Feb. 23, 2023

(54) CLAVICULAR BONE PLATE

(71) Applicant: Bonebridge AG, Zug (CH)

(72) Inventors: Patrick BURKI, Solothurn (CH); Christian GERBER, Zug (CH); Beat Kaspar MOOR, Venthône (CH)

(21) Appl. No.: 17/405,565

(22) Filed: Aug. 18, 2021

(51) Int. Cl. A61B 17/80

(52) U.S. Cl. CPC A61B 17/8061; A61B 17/8085; A61B 17/8052

(57) ABSTRACT

A bone plate (1) for positioning along a medial or lateral portion of a clavicle including an upper surface (2) which, in an operative position, faces away from the clavicle, a lower surface (3) which, in the operative position, faces toward the clavicle, a non-linear central longitudinal axis (4), a middle portion (5) of the length L_{M}, a left terminal portion (6) of the length L_{L} extending from the left end of the middle portion having a plurality of circular screw holes (8) for receiving bone screws, and a right terminal portion (7) of the length L_{R} extending from the right end of the middle portion having a plurality of circular screw holes (9) for receiving bone screws. The total length L of the bone plate corresponds to L=L_{M}+L_{L}+L_{R}, and at least one of the circular screw holes is a variable angle screw hole.

### BACKGROUND OF INVENTION

### Field of Invention

The present invention generally relates to a clavicle bone plate.

### Brief Description of Related Art

Between 2.6% and 5% of all fractures affect the clavicle, which makes it one of the most frequently broken bones of the human skeleton. Most frequently the middle third of the clavicle is affected (69-82%). Lateral fractures account for about 15% and medial fractures are rare (2-5%). The majority of the fractures are either simple and non-displaced or simple and displaced. About 20% of the fractures are comminuted fractures mostly due to high energy trauma (particularly from sports). Even though fractures of the clavicle were considered a domain for non-operative treatment, recent studies have shown that the rate of malunion or non-union after non-operative treatment might be well higher than previously shown.

A clavicle plate is known from EP-B 2 355 730 which has a straight central axis and a number of screw holes for receiving bone screws at a predetermined non-variable angle.

One problem associated with the above described clavicle plate is its straight shape which makes it necessary to adapt the plate pre- or intra-operatively to the anatomical conditions. A further problem is due to the non-variability of the angle of introduction of the bone screws through the screw holes.

Thus, there remains a need for an improved clavicle bone plate for properly positioning fractured bone fragments using variable angle bone screws and allowing to use a single plate for both the right and the left clavicle, and which allows to overcome the above described disadvantages of prior art clavicle plates.

### BRIEF SUMMARY OF THE INVENTION

The present invention relates to a clavicle bone plate sized and shaped to be positioned along a medial or lateral portion of a clavicle shaft and comprises:
(i) an upper surface (2) which, in an operative position, faces away from the clavicle bone;
(ii) a lower surface (3) which, in the operative position, faces toward the clavicle bone;
(iii) a non-linear central longitudinal axis (4);
(iv) a middle portion (5) of the length L_{M};
(v) a left terminal portion (6) of the length L_{L} extending from the left end of the middle portion (5) having a plurality of first circular screw holes (8) for receiving bone screws; and
(vi) a right terminal portion (7) of the length L_{R} extending from the right end of the middle portion (5) and having a plurality of second circular screw holes (9) for receiving bone screws;
wherein:
(vii) the total length L of the bone plate (1) corresponds to L=L_{M}+L_{L}+L_{R}; and
(viii) at least one of the first or second circular screw holes (8;9) is a variable angle screw hole which allows to receive a bone screw at a user-selected angle.

The clavicle bone plate according to the invention is intended for use in internal fixation in the clavicle. It is indicated for:

fixation of fractures of the clavicle shaft;

fixation of fractures of the lateral clavicle;

malunions of the clavicle; and

non-unions of the clavicle.

One of the advantages of the clavicle bone plate according to the invention is that the plate according to the invention has two opposite curvatures which are adjusted so that the plate is matched to lateral as well as medial shaft fractures of the clavicula. For lateral fractures, which are the majority, the plate is placed so that the side of the plate with more holes is placed laterally. Now, if one has a medial lying shaft fracture, one can simply rotate the same plate 180° and place it further medially. This is a significant advantage. The plate constriction is so that the surgeon can adjust the curvature of the plate with an instrument. This is a further important advantage.

Further advantageous embodiments of the invention can be commented as follows:

In a special embodiment the middle portion of the bone plate is free of screw holes.

According to another embodiment of the invention the left terminal portion of the bone plate is curved in one direction and has a number x of first circular screw holes and the right terminal portion is curved in an opposite direction to the curvature of the left terminal portion and has a number y of second circular screw holes, where y<x.

According to a further aspect of the invention the centers of the first and second circular screw holes are arranged along the non-linear central longitudinal axis. This allows to minimize the width of the bone plate (compared to a bone plate with staggered screw holes).

According to a further aspect the bone plate as seen in a lateral view thereof is curved.

Still to another aspect of the invention the length L_{M} of the middle portion measures at least 0.15 L, preferably at least 0.20 L. The length L_{L} of the e left terminal portion may correspond to least 0.35 L, preferably at least 0.40 L. The length L_{R} of the right terminal portion may correspond to at least 0.3 L, preferably at least 0.35 L.

According to a further aspect of the invention the curved left terminal portion of the bone plate has a radius of curvature in the range of 50 mm-80 mm. The curved right terminal portion may have a radius of curvature in the range of 85 mm-115 mm.

According to another aspect of the invention the width of the left terminal portion is widening from the middle portion to the free end of the left terminal portion.

The left terminal portion may have a number of first circular screw holes which is at least twice as much as the number of second circular screw holes of the right terminal portion.

According to a further aspect of the invention the middle portion may comprise at least one suture hole extending transverse to the longitudinal axis for receiving a suture. That suture hole allows to perform suture cerclage of the clavicle plate.

According to a further aspect of the invention the at least one of the first or second circular screw holes (8;9) is a variable angle screw hole configured to receive a bone screw at an angle of ±15° with regard to the central axis of the screw hole. The at least one of the first or second circular screw holes may have an inner wall made of a material which has a hardness H_{P}, with a hollow cylinder-shaped or hollow cone-shaped insert mounted in the screw hole, which least partially abuts the inner wall and is suitable for accommodating the head of a bone screw. The insert is arranged in the at least one of the first or second circular screw holes such that it is secured against rotation. The insert consists of a material that has a hardness H_{E}<H_{P}.

The central longitudinal axis of the bone plate may be essentially straight in the middle portion of the bone plate.

The at least one of the left or right terminal portions of the bone plate may be provided with a long hole, preferably allowing compression in both directions. The long hole has the advantage to allow interfragmentary compression where needed.

According to a further aspect the bone plate may comprise at least one bending zone with a reduced width between two adjacent first and/or second circular screw holes.

The lower surface of the bone plate—as seen in a cross-section orthogonal to the central longitudinal axis—may be concave, preferably cylindrically concave.

The upper surface of the bone plate—as seen in a cross-section orthogonal to the central longitudinal axis—may be convex, preferably cylindrically convex.

According to a further aspect of the invention the cross-section of the bone plate orthogonal to central longitudinal axis of the bone plate may taper in a trapezoidal shape in the direction of the lower surface.

In special embodiment of the invention all of the first and second circular screw holes of the bone plate are variable angle screw holes.

According to a further special embodiment the bone plate comprises at least one, preferably at least two K-wire holes of lesser diameter compared to the diameter of the first and second circular screw holes and which is preferably parallel to one of the first or second circular screw holes (8;9). This allows to receive a Kirschner wire for defining an optimal screw alignment, for fixing the plate to the bone, the fracture to be reduced and held and also to guarantee the reposition of the bone fracture.

The bone plate according to the invention may be designed also in a mirror inverted version.

### BRIEF DESCRIPTION OF THE DRAWINGS

Several embodiments of the invention will be described in the following by way of example and with reference to the accompanying drawings in which:

FIG. 1 illustrates a perspective view of a special embodiment of a midshaft clavicle bone plate according to the invention;

FIG. 2 illustrates a top view of the clavicle plate according to FIG. 1;

FIG. 3 illustrates a lateral view of the clavicle plate according to FIG. 1;

FIG. 4 illustrates a perspective side view of the clavicle plate according to FIG. 1;

FIG. 5 illustrates a perspective view of another embodiment of a midshaft clavicle bone plate according to the invention;

FIG. 6 illustrates a top view of the clavicle plate according to FIG. 5;

FIG. 7 illustrates a lateral view of the clavicle plate according to FIG. 5;

FIG. 8 illustrates a perspective side view of the clavicle plate according to FIG. 5;

FIG. 9 illustrates a perspective view of an embodiment of a lateral clavicle bone plate according to the invention;

FIG. 10 illustrates a top view of the clavicle plate according to FIG. 9;

FIG. 11 illustrates a lateral view of the clavicle plate according to FIG. 9;

FIG. 12 illustrates a perspective side view of the clavicle plate according to FIG. 9;

FIG. 13 illustrates a perspective view of another embodiment of a lateral clavicle bone plate according to the invention;

FIG. 14 illustrates a top view of the clavicle plate according to FIG. 13;

FIG. 15 illustrates a lateral view of the clavicle plate according to FIG. 13; and

FIG. 16 illustrates a perspective side view of the clavicle plate according to FIG. 13.

### DETAILED DESCRIPTION OF THE INVENTION

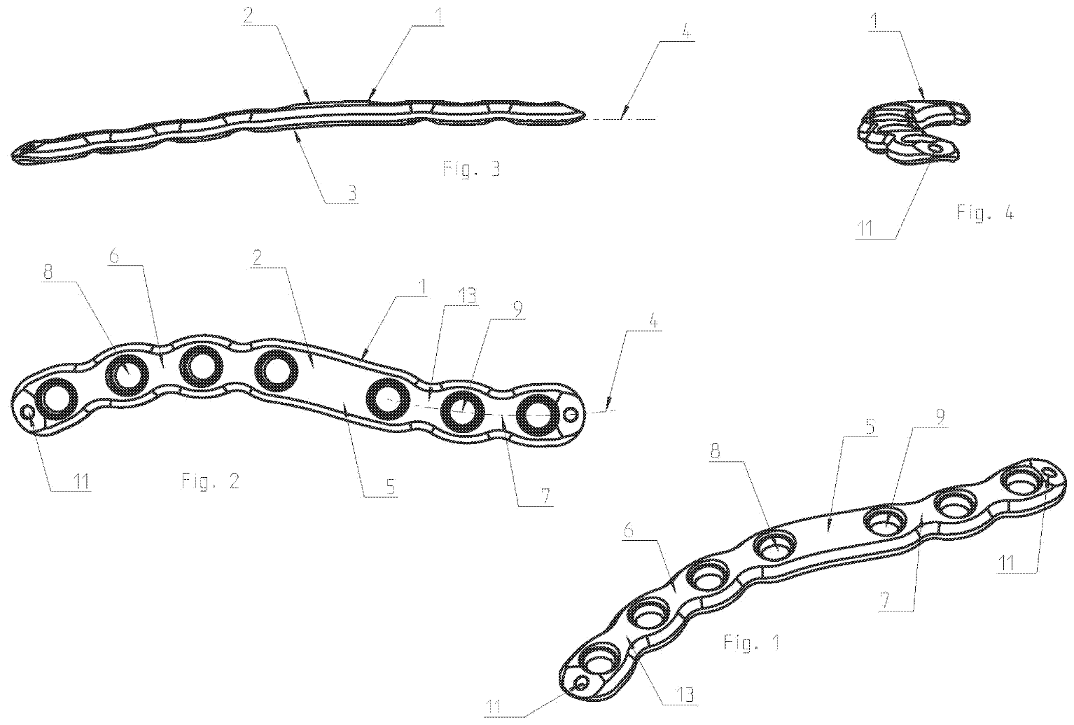

FIGS. 1 to 4 illustrate a first embodiment of a midshaft clavicle bone plate 1 according to the invention. The bone plate 1 has an upper surface 2 which, in an operative position, faces away from the clavicle bone and a lower surface 3 which, in the operative position, faces toward the clavicle bone. The bone plate 1 has a longitudinal axis 4 which basically has an S-shape. The middle portion 5 of the length L_{M} of the bone plate is essentially straight. A left terminal portion 6 of the length L_{L} extends from the left end of the middle portion 5 and has four first circular screw holes 8 for receiving bone screws.

A right terminal portion 7 of the length L_{R} extends from the right end of the middle portion 5 and has three second circular screw holes 9 for receiving bone screws.

The left terminal portion 6 is curved in one direction and the right terminal portion 7 is curved in an opposite direction to the curvature of the left terminal portion 6 so that the longitudinal axis 4 basically has an S-shape.

The total length L of the bone plate 1 therefore corresponds to L=L_{M}+L_{L} L_{R}.

All of the seven of the first and second circular screw holes 8;9 are variable angle screw holes which allow to receive a bone screw at a user-selected angle and are arranged along the central longitudinal axis 4.

The variable angle screw holes 8;9 allow to receive a bone screw at an angle of ±15° with regard to the central axis of the screw hole. The screw holes 8;9 have an inner wall made of a material which has a hardness H_{P}. A hollow cylinder-shaped or hollow cone-shaped insert is mounted in each screw hole, which at least partially abuts the inner wall and is suitable for accommodating the head of a bone screw. The insert is arranged in the screw hole such that it is secured against rotation. The insert consists of a material that has a hardness H_{E}<H_{P}.

Between individual first and/or second circular screw holes (8;9) there are bending zones 13 with a reduced plate width allowing to adapt the shape of the bone plate 1 more easily.

The lower surface 3 of the bone plate 1—as seen in a cross-section orthogonal to the central longitudinal axis 4—is cylindrically concave, whereas the upper surface 2—as seen in a cross-section orthogonal to the central longitudinal axis 4—is cylindrically convex. The cross-section of the bone plate 1 orthogonal to its central longitudinal axis 4 tapers in a trapezoidal shape in the direction of the lower surface 3.

The free ends of the left terminal portion 6 and of the right terminal portion 7 are beveled and comprise each a Kirschner wire hole 11 which is parallel to the first circular screw holes 8, and the second circular screw holes 9, respectively. This allows to define an optimal screw alignment, for fixing the plate to the bone, the fracture to be reduced and held and also to guarantee the reposition of the bone fracture.

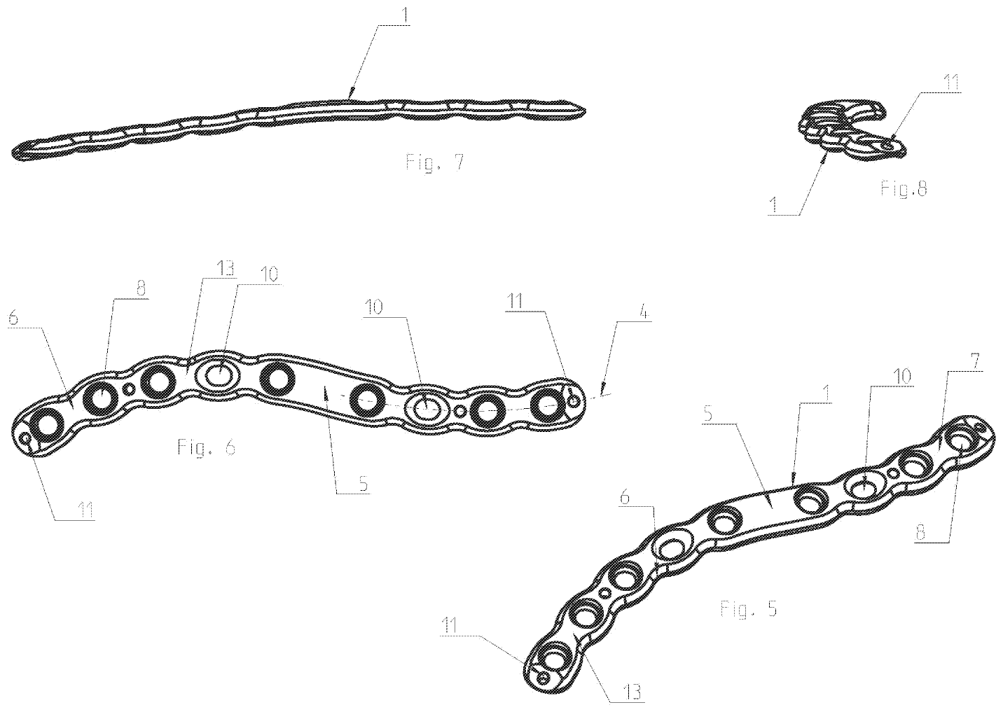

FIGS. 5 to 8 illustrate a second embodiment of a midshaft clavicle bone plate 1 according to the invention. With respect to the first embodiment according to FIGS. 1-4 the following modifications are present. The left terminal portion 6 as well as the right terminal portion 7 are both provided with a long hole 10 allowing interfragmentary compression in both directions of the longitudinal axis 4 where needed. Furthermore, the plate 1 is provided with four K-wire holes 11 for receiving Kirschner wires.

The long hole 10 in the left and right terminal portions 6;7 are positioned between the most central first circular screw hole 8 and second most central first circular screw hole 8. Two of the K-wire holes are positioned at the left and the right end of the plate 1 and the two other K-wire holes are positioned centrally of the second last first circular screw holes.

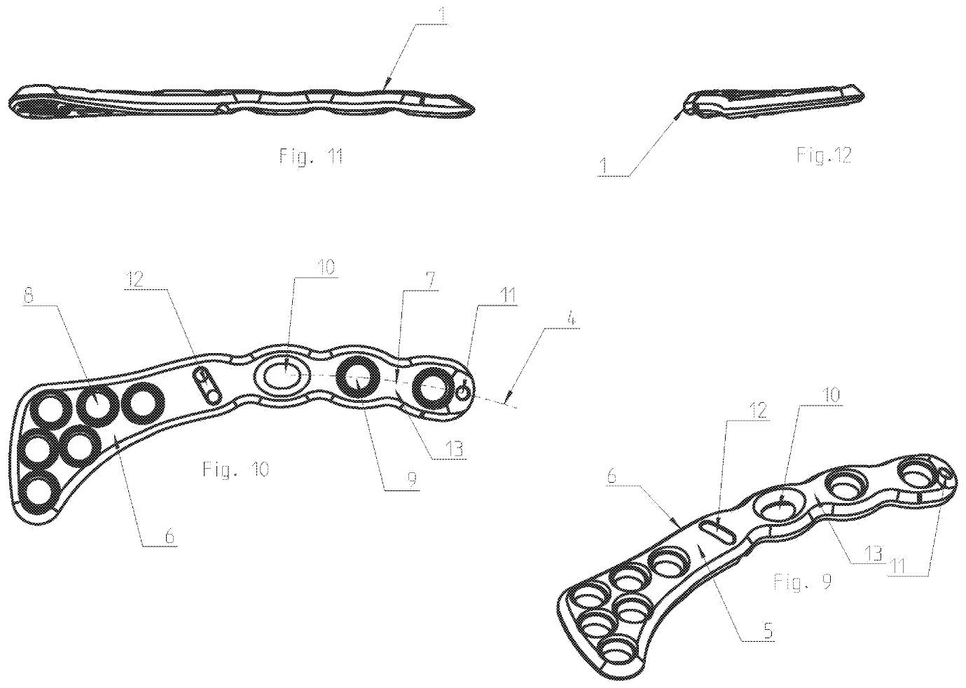

FIGS. 9 to 12 illustrate a first embodiment of a lateral clavicle bone plate 1 according to the invention. Compared to the two embodiments of the midshaft clavicular plates according to FIGS. 1-4 and 5-8 the left terminal portion 6 of the bone plate 1 is enlarged towards the left end and is provided with a larger number (six) of first circular screw holes 8. A K-wire hole 11 is provided at the beveled free end of the right terminal portion 7.

A single long hole 10 is positioned in the right terminal portion 7 and the middle portion 5 is provided with a suture hole 12 extending transverse to the longitudinal axis 4 for receiving a suture allowing a suture cerclage of the clavicle plate. The surgeon can make a suture cerclage with the suture hole 12. For example, a suture anchor can be screwed into the coracoid and then the sutures are looped through the suture hole and then tightened with a knot so that the position of the clavicle can be secured.

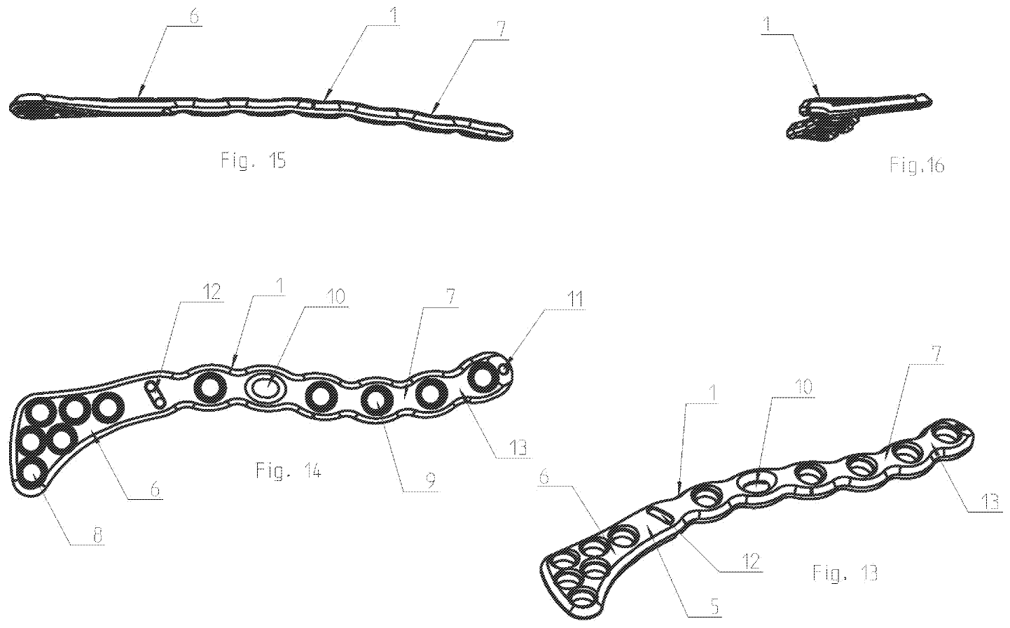

FIGS. 13 to 16 illustrate a second embodiment of a lateral clavicle bone plate 1 according to the invention. It is basically similar to the first embodiment according to FIG. 9-12 with the exception of the right terminal portion 7 which is longer and provided with four first circular screw holes 8 (instead of only two).

### Fracture Reduction and Temporary Plate Fixation

Restoration of normal length, axis and rotation remains the fundamental principle for fracture reduction in shaft fractures. After exposing the clavicle, the two fragments may have to be distracted to restore the length. The properly chosen plate may be placed on the clavicle to assist in temporary fixation. A reduction forceps may be used to allow for proper reduction; furthermore, large fragments may also be reduced and held temporarily in place with bone clamps or Kirschner wires. A usual plate holding and reduction clamp for the clavicle may be used to facilitate temporary fixation with the plate in place. Kirschner wire holes available at the lateral and medial ends of the clavicle bone plate according to the invention further assist with temporary maintenance of the reduction.

The clavicle bone plate may be used as internal fixator to allow for bridging osteosynthesis. Only the main fragments are being reduced while the comminuted fracture zone is left without screws to allow for indirect fracture healing.

For very lateral Neer Type IIB clavicle fractures, indirect fixation of the fracture with suture fixation (through the specific suture hole(s) provided in the lateral clavicle bone plate according to the embodiment of FIGS. 9-12 and 13-16 reduce the coraco-clavicular dehiscence and allow for healing.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

What is claimed is:

1: A bone plate for positioning along a medial or lateral portion of a shaft of a clavicle bone, the bone plate comprising:
(i) an upper surface which, in an operative position, faces away from the clavicle bone;
(ii) a lower surface which, in the operative position, faces toward the clavicle bone;
(iii) a non-linear central longitudinal axis;
(iv) a middle portion having a length L_{M};
(v) a left terminal portion having a length L_{L} extending from a left end of the middle portion, said left terminal portion having a plurality of first circular screw holes for receiving bone screws;
(vi) a right terminal portion of the length L_{R} extending from a right end of the middle portion and having a plurality of second circular screw holes for receiving bone screws;
wherein
(vii) a total length L of the bone plate corresponds to L=L_{M}+L_{L}+L_{R}; and
(viii) at least one of the first or second circular screw holes is a variable angle screw hole configured to receive a bone screw at a user-selected angle.

2: The bone plate according to claim 1, wherein the middle portion is free of screw holes.

3: The bone plate according to claim 1, wherein the left terminal portion is curved in one direction and has a number x of first circular screw holes, and wherein the right terminal portion is curved in an opposite direction to the curvature of the left terminal portion and has a number y of second circular screw holes, where y<x.

4: The bone plate according to claim 1, wherein centers of the first and second circular screw holes are arranged along the non-linear central longitudinal axis.

5: The bone plate according to claim 1, wherein the bone plate as seen in a lateral view thereof is curved.

6: The bone plate according to claim 1, wherein the length L_{M} of the middle portion measures at least 0.15 L.

7: The bone plate according to claim 1, wherein the length L_{L} of the left terminal portion measures at least 0.35 L.

8: The bone plate according to claim 1, wherein the length L_{R} of the right terminal portion measures at least 0.3 L.

9: The bone plate according to claim 3, wherein the curved left terminal portion has a radius of curvature in the range of 50 mm-80 mm.

10: The bone plate according to claim 3, wherein the curved right terminal portion has a radius of curvature in the range of 85 mm-115 mm.

11: The bone plate according to claim 1, wherein the width of the left terminal portion increases from the middle portion to a free end of the left terminal portion.

12: The bone plate according to claim 1, wherein the left terminal portion has a number x of the first circular screw holes which is at least twice as much as a number y of the second circular screw holes of the right terminal portion.

13: The bone plate according to claim 1, wherein the middle portion comprises at least one suture hole extending transverse to the non-linear central longitudinal axis for receiving a suture.

14: The bone plate according to claim 1, wherein at least one of the first or second circular screw holes is a variable angle screw hole that is configured to receive a bone screw at an angle of ±15° with regard to a central axis of the variable angle screw hole.

15: The bone plate according to claim 1, wherein at least one of the first or second circular screw holes has an inner wall made of a material which has a hardness H_{P}, wherein a hollow cylinder-shaped or hollow cone-shaped insert is mounted in said at least one of the first or second circular screw holes such that said insert at least partially abuts the inner wall, wherein the insert is configured to accommodate a head of a bone screw, wherein the insert is arranged in said at least one of the first or second circular screw holes such that it is secured against rotation, and wherein the insert consists of a material that has a hardness H_{E}<H_{P}.

16: The bone plate according to claim 1, wherein the non-linear central longitudinal axis is essentially straight in the middle portion.

17: The bone plate according to claim 1, wherein at least one of the left or right terminal portions is provided with a long hole.

18: The bone plate according to claim 1, wherein the bone plate comprises at least one bending zone having a reduced width between two adjacent first or second circular screw holes.

19: The bone plate according to claim 1, wherein the bone plate comprises at least one K-wire hole of lesser diameter compared to the diameter of the circular screw holes.

20: A bone plate that is a mirror image version of a bone plate according to claim 1.
